(19) United States
(12) Patent Application Publication
(10) Pub. No.: US 2021/0011020 A1
(43) Pub. Date: Jan. 14, 2021

(54) INTEGRIN ALPHA V AS A PROTEIN MARKER FOR DIAGNOSING PROSTATE CANCER

(71) Applicant: LIMITED LIABILITY COMPANY "PROSTAGNOST" [RU/RU], Moscow (RU)

(72) Inventors: Marina Yurievna ZEMSKOVA, Pushchino (RU); Varlam Borisovich Keshelava, Moscow (RU); Konstatin Sergeevich Sorokin, Korolev (RU)

(73) Assignee: LIMITED LIABILITY COMPANY "PROSTAGNOST" [RU/RU], Moscow (RU)

(21) Appl. No.: 16/504,343

(22) Filed: Jul. 8, 2019

(51) Int. Cl. G01N 33/574; G01N 33/493; G01N 33/68; G01N 33/50; G16B 40/10

(52) U.S. Cl. CPC G01N 33/57434; G01N 33/493; G16B 40/10; G01N 33/5011; G01N 33/6827

(57) ABSTRACT

The invention relates to the field of biology and medicine, specifically to oncology, and can be used for diagnosing prostate cancer. For this purpose, use is made of a novel prostate cancer marker, which can be identified in the urine of a patient and constitutes of integrin-alpha V level or a fragment thereof. A method of diagnosing prostate cancer, comprising identifying integrin-alpha V in the urine of a patient, and a kit for carrying out the diagnosis, comprising at least one antibody specific to integrin-alpha V, or a functional fragment thereof, are also proposed. The invention makes it possible to carry out a non-invasive diagnosis of prostate cancer using a simple, sensitive and reliable method, which can be used under standard clinical laboratory conditions in inpatient or outpatient medical institutions.

The present application is a continuation of PCT/RU2017/000386 filed on Jun. 2, 2017. The content of the abovementioned application is incorporated by reference herein.

### SEQUENCE LISTING

The instant application contains a Sequence Listing which has been filed electronically under name “Sequence_listing” on Jul. 8, 2019 in ASCII format and contains 9 kB.

### FIELD OF THE INVENTION

The invention relates to the field of biology and medicine, particularly, oncology, and can be used for diagnosis of prostate cancer, as well as for monitoring of the disease and treatment efficacy.

### BACKGROUND OF THE INVENTION

After skin cancer, prostate cancer (PC) is the most commonly diagnosed form of cancer. In many countries PC is the second/third most common cause of death for men over the age of 40 years. Currently it is believed that early diagnosis is the most important factor in fight against PC.

An obvious way to identify tumor markers is to search for substances that have significantly different concentration in fluids of cancer patients compared to healthy people. The traditional and most common approach of detecting PC is the PSA test (prostate specific antigen, PSA) in serum (Stamey T A, Yang N, Hay A R, McNeal J E, Freiha F S, Redwine E. Prostate-specific antigen as a serum marker for adenocarcinoma of the prostate. N Engl J Med. 1987 Oct. 8; 317(15):909-16). However, during over 30 years of clinical practice a large amount of statistical data has been accumulated, indicating a low serum PSA specificity as a tumor marker, which manifests itself in the form of a significant number of false-positive results. Therefore, attention of many researchers has focused on finding new tumor markers.

Currently, a number of prostate cancer biomarkers (for example PCA3, TMPRSS2: ERG, PSCA (prostate stem cell antigen), AMCAR) are being actively studied. The best of them utilize non-invasive techniques by measuring the quantities of these markers in urine samples (see, for example, Koo K. M. et al., Colorimetric TMPRSS2-ERG Gene Fusion Detection in Prostate Cancer Urinary Samples via Recombinase Polymerase Amplification//Theranostics. 2016 Jun. 15; 6 (9): 1415-24; Deras I. L. et al., PCA3: a molecular urine assay for predicting biopsy outcome//J Urol. 2008 April; 179 (4): 1587-92). However, these markers require use of rather expensive methods based on polymerase chain reaction for their detection, and these analyses are difficult to produce in the framework of a standard clinical laboratory.

Recently, the possibility of diagnosing prostate cancer using biomarkers of urine exosomes has been actively investigated. For example, patent U.S. Pat. No. 7,897,356 B2 discloses a method for diagnosing prostate cancer by identifying a biosignal of exosomes, comprising capturing and isolating exosomes using surface exosome proteins CD63, CD9 or CD81, and then performing a qualitative or quantitative analysis of the level of specific PC markers PSMA, PCSA, EpCam, B7H3 (CD276). Another proposed method and a kit for quantifying and determining exosomes for diagnosing prostate cancer and prostate hyperplasia (US 20130196355 A1) is based on an enzyme immunoassay using anti-Rab5 as a trapping antibody to isolate all the exosomes, from which subsequently exosomes are isolated using anti-PSA detecting antibodies and the level of these exosomes is compared to the reference level. However, firstly, a fast and non-invasive biomarker suitable for implementation of clinical laboratory diagnostics of prostate cancer on a standard laboratory equipment was never found; and secondly, isolation of exosomes, which is necessary for realization of the mentioned methods, is a very nontrivial task that cannot be adequately solved by majority of clinical laboratories.

Thus, despite existing studies on the diagnosis of prostate cancer, medical practice still needs a new routine method. This method should be non-invasive, fast, simple and use equipment that is common in clinical laboratories. This type of method can also be used for routine/sequential analysis in studies such as population surveys, monitoring risk groups, monitoring relapses, monitoring the course of the disease, monitoring the effectiveness of therapy etc.

Modern immunochemical methods make it possible to quantify low concentrations of target proteins directly in complex biological fluids. The difference in the measurement methods for different antigens is in using different necessary antibodies but required procedures are similar and well known. This versatility of such methods has led to widespread use of these approaches in clinical laboratories.

### SUMMARY OF THE INVENTION

The task and the technical result of the invention is to find a new diagnostic marker for PC, allowing for a non-invasive diagnosis of the disease, as well as developing on its basis a simple, sensitive, reliable method for diagnosis of PC suitable for conventional clinical laboratories in hospitals and medical institutions.

The present invention relates to the use of integrin alpha V (alone or in combination with other markers) or its fragment detected in urine as a biomarker of prostate cancer. According to the invention, the integrin alpha V or its fragment can be used as a biomarker by identifying them as an individual subunit and as a part of integrin heterodimers, such as αvβ1, αvβ3, αvβ5, αvβ6 and/or αvβ8.

In particular, the problem is solved by using a noninvasive method for distinguishing prostate cancer from a healthy state, comprising: (1) collecting a sample of a first portion of a urine from the subject; (2) determining levels of integrin alpha V (ITGAV) or its fragments in the sample; (3) comparing these levels with a reference value, obtained from urine samples collected from non-cancer subjects and processed in essentially the same way as the subject's sample; (4) predicting presence of prostate cancer in said subject when measured levels are lower than the reference value; predicting an absence of prostate cancer in said subject when said levels are higher than said reference value.

In some realizations of the invention, the noninvasive method as described above is further characterized by the fact that the level of integrin alpha V or its fragments was determined in the first portion of the morning urine from the subject without additional processing of said first portion. Term “without additional processing” indicates that the urine sample may be directly diluted in water or buffer suitable for determining the level of integrin alpha V or its fragments in the sample, and no additional processing steps are required. For example, there is no need for lysis of the sample, high speed centrifugation of the sample or fractionation of extracellular vesicles from the sample.

In a preferred realization of the invention, the level of integrin alpha V or its fragments is determined by an enzyme-linked immunosorbent assay. In another preferred realization of the invention, the first portion comprises between 25-30 ml of the first morning urine. In yet another realization of the invention, the human subject is a subject who was never treated for prostate cancer.

In some realizations of the invention, a method for treating prostate cancer in a human subject is presented, comprising:
obtaining a sample of a first portion of a morning urine from the subject;
determining a level of integrin alpha V or its fragments in said sample;
comparing said level with a reference value, obtained from urine samples collected from non-cancer subjects and processed in essentially the same way as the subject's sample;
when said level is less than said reference value, referring said subject to one or more additional methods for prostate cancer verification or classification;
treating the subject by one or more available methods based on the verification or classification outcome.

As to absolute amounts of ITGAV or its fragments per ml of a body fluid, preferably the first portion of urine, it is preferably around between 7.8-20.0 ng/ml (or 1.95-5.0 ng/ml in 1:4 dilution of a sample), more preferably around between 7.9-16.0 ng/ml (or 1.975-4.0 ng/ml in 1:4 dilution) for control samples. For cancer samples, preferred ranges are around between 0.02-8.0 ng/ml (or 0.005-2.05 ng/ml in 1:4 dilution).

In terms of absolute amounts of ITGAV or its fragments per ml of a body fluid, preferably first portion of urine, this is preferably around between 98-199% of reference level, more preferably around between 99-180% of reference level, and most preferably around 100-150% of reference level for control samples. For cancer samples, preferred ranges are around between 1-99% of the reference level.

The reference value used in the noninvasive method according to this invention is determined as follows: more than 92%, preferably 93%, preferably 94%, preferably 95%, preferably 96%, preferably 97%, preferably 98%, preferably 99%, preferably 99.5%, preferably 99.9%, preferably 100% patients with PC have said biomarker level under said reference value, and more than 92%, preferably 93%, preferably 94%, preferably 95%, preferably 96%, preferably 97%, preferably 98%, preferably 99%, preferably 99.5%, preferably 99.9%, preferably 100% of non-cancer subjects have said biomarker level over said reference value. The exact value of this reference value may vary for patients of different races or a mixed race. The reference value may also vary when a different quantification method is used.

The term “diagnosis” encompasses identification, confirmation, and or characterization of the presence of absence or absence of prostate cancer.

The words “processed in essentially the same way” mean that the urine samples collected from non-cancer subjects are processed to determine the reference value in a similar manner, utilizing similar method steps, as the subject's sample. There may be small deviations or differences between the two procedures (such as analogous but different reagents), but these deviations and/or differences should not result in the difference in the integrin alpha V or its fragment levels.

In some realizations of the invention, the noninvasive method as described above is further characterized by the fact that a processed first portion of urine from the subject is used for determination of integrin alpha V or its fragments levels.

The preferred type of urine processing in this case is a low-speed centrifugation (such as, for example, at a speed of 300-500 g for 10-15 minutes) or filtration through membranes with a pore size of 0.22-0.45 nm, or any other available method for separation of the soluble fraction of urine proteins from the insoluble sediment. The sediment is discarded after processing. In other realizations of the invention, the first portion of urine from the subject can be used directly without processing.

In some realizations of the invention, the noninvasive method as described above is further characterized by the fact that the human subject was never treated for prostate cancer. Exclusion of subjects previously treated from prostate cancer may increase specificity of the method according to the present invention. Subjects with an altered prostate (for example, removed because of surgical procedure) may show a reduction in the level of integrin alpha V that is not related to the presence of cancer. Also, chemotherapy may result in unpredictable changes in the level of integrin alpha V.

In some realizations of the invention, the noninvasive method as described above is further characterized by the fact that the first portion comprises up to 30 ml of the first urine portion.

In some realizations of the invention, the noninvasive method as described above is further characterized by the fact that the human subject is a Caucasian and the reference value is 8 ng/ml.

In some realizations of the invention, the noninvasive method as described above is further characterized by the fact that the level of integrin alpha V or its fragments is determined by an enzyme-linked immunosorbent assay or by any other method of the protein identification (e.g. mass spectrometry, western blotting, lateral flow assay, etc.). For example, detection and quantification of integrin alpha V or a fragment thereof may be performed by one of the following techniques: MALDI-TOF, SELDI, via interaction with a ligand or ligands, 1-D or 2-D gel-based analysis systems, Liquid Chromatography combine liquid chromatography and Mass spectrometry techniques including ICAT® or iTRAQ®, thin-layer chromatography, and NMR spectroscopy, or possibly by combination of the above mentioned techniques.

In some realizations of the invention, the noninvasive method as described above is further characterized by the fact that the level of integrin alpha V is determined as individual subunit or one of the integrin heterodimers selected from the following group: αvβ1, αvβ3, αvβ5, αvβ6, αvβ8. Also, total integrin alpha V level may be determined in the mix of αvβ1, αvβ3, αvβ5, αvβ6, αvβ8.

In some realizations of the invention, additional methods of prostate cancer verification or classification may be used including palpation, transrectal ultrasound examination (or scan), multi-point biopsy with histological examination or other. Additional clinical, laboratory and/or instrumental examinations may be used to verify the PC diagnosis and its stage.

Based on the results or outcome of the verification or classification, an appropriate treatment may be prescribed. The exact treatment will be chosen by a physician based on the stage of the disease and available practices. For example, in the case of prostatitis or benign prostatic hyperplasia a change of lifestyle and/or some specific drug may be prescribed; for an advanced benign prostatic hyperplasia or a localized oncology, a partial removal of the affected tissue may be prescribed; for an advanced PC, a radical prostatectomy (complete removal of the prostate) and chemotherapy is usually an option of choice.

The problem is also solved by developing a kit for diagnosing prostate cancer in the patient's whole urine, which includes at least one antibody or its functional fragment specific for alpha V integrin or its fragment and not cross-specific to other urine peptides. In some realizations of the invention, the kit is a test system for conducting enzyme-linked immunosorbent assay.

### BRIEF DESCRIPTION OF THE DRAWINGS

FIG. 1. Anatomical section of human urinary system, showing prostate gland and urethral canal.

FIG. 2. An example of a sampling device to obtain separated portions of urine sample from a subject. The contact urinal receiver (2.2) is made of a soft polymeric material that does not cause discomfort during a contact with the head of the penis. Urine entering the urinal receiver and moving freely through the main channel (2.3) is collected in the Uroflowmeter (2.1), which records the dynamics of fluid accumulation. A flexible polymer tube (2.4) made of a biologically inert material is attached to a side channel. The speed of a peristaltic pump (2.5) that regulates operation of the device is chosen so that in the process of urination a segment of the tube suitable for further applications will be filled.

FIG. 3. Comparison of ITGAV (A) and IGFBP7 (B) concentrations between the first (#n-1) and second (#n-2) portions of urine from healthy individuals. The volume of first portions was approximately 30 ml of first morning urine, while the second portion represents the rest of the urine obtained during urination (volumes varied). The difference in values obtained from between the first and second portion of urine for ITGAV is statistically significant (p<0.05) while there is no difference for IGFBP7 protein level (p>0.05) for values in both portions.

FIG. 4. The ITGaV level is decreased in the first portions of urine samples taken from of PC patients as compared to the non-cancer group. N is the number of subjects in the groups (n=12, 25, 25 for normal, adenoma (BPH) and PC, respectively). The horizontal line shows a relative ITGaV level to distinguish PC. A.v.—arbitrary value.

FIG. 5. The results of mathematical processing of the data shown in FIG. 4. X axis represents the relative level of ITGaV. Y-axis represents the probability of detecting integrin alpha V in the urine of various groups of subjects. Vertical lines show mathematical expectations. A.v.—arbitrary value.

FIG. 6. The results of measuring the level of integrin alpha V in the first portion of urine in healthy subjects and individuals diagnosed with PC using a larger cohort of samples (n for normal=22; n for adenoma (BPH)=42 and n for PC=47 respectively). The volume of first portions was approximately 30 ml of first morning urine. A 25 μl of urine was used in the assay. Using a 8 ng/ml cutoff, ITGaV level was decreased below the reference value in 43 of 47 (91.5%) of the samples from men with PC confirmed by biopsy.

FIG. 7. ROC curve for the ITGAV values shown on FIG. 6 demonstrating that urinary ITGAV was capable of distinguishing PC from benign conditions with a high sensitivity of 91.5% and specificity of 91.4%.

### DETAILED DISCRIPTION OF THE INVENTION

For better understanding of this invention, some terms used herein are set forth below. In the description of this invention, the terms “include” and “including” are deemed as meaning “includes, among other things”. These terms are not intended to be interpreted as “consists only of”.

The term PC refers to prostate cancer, BPH refers to benign prostate formations (adenoma, prostatitis) and “normal” samples refers to samples obtained from individuals over 50 years old without pathologies of the prostate gland.

The term “whole urine” refers to urine samples that are not subjected to any processing, or soluble samples obtained after a low-speed centrifugation, filtration, or other procedures that allow the separation of soluble urine components from the insoluble fraction.

“Marker” or “biomarker”, unless otherwise specified, in the present description mean the following: integrin alpha V (identification number UniProtKB P06756-1; HGNC: 6150; Entrez Gene: 3685; Ensembl: ENSG00000138448; OMIM: 193210) and its isoforms (P06756-2; P06756-3). Integrin alpha V (other names αV, CD51, MSK8, vitronectin receptor α (VNRα)) is a membrane protein, a glycoprotein from the integrins superfamily, the ITGaV gene product located on the 2nd chromosome at position 2q31-q32.

Integrin alpha V consists of 1048 amino acids. The mature integrin alpha V molecule is a glycoprotein with a length of 1018 amino acids (see SEQ ID NO: 1), the molecular weight of the protein portion is 116.0 kDa.

A mature integrin alpha V molecule forms a heterodimeric complex with beta-1, beta-3, beta-5, beta-6 or beta-8 integrins. The alpha V integrins are receptors for a number of ligands, recognizing the sequence of arginine-glycine-aspartate (R-G-D). Integrins with the alpha V subunit are involved in many processes of embryo development, in angiogenesis and osteoporosis.

Where reference is made herein to a particular biomarker name or sequence, such as a reference sequence or a Sequence ID NO, this applies to all Sequence ID NOs and any of the integrin alpha V sequences provided below, unless otherwise apparent.

Preferably, the fragment comprises at least 80% sequence homology to the reference sequence (e.g. HGNC: 6150; Entrez Gene: 3685; Ensembl: ENSG00000138448; OMIM: 193210; UniProtKB: P06756), more preferably 85%, more preferably 90%, more preferably 95%, more preferably 97%, more preferably 99% and most preferably 99.9% sequence homology to the reference sequence, or as close thereto as appropriate. Suitable methods for establishing sequence homology include the BLAST programme.

Preferably, the fragment comprises at least four consecutive amino acids from the reference sequence, more preferably at least 5, more preferably at least 6, more preferably at least 7, more preferably at least 8 consecutive amino acids from the reference sequence, although longer fragments at least 10, 15, 20, 25, 30, 40, 50, 60, 80, 100 and up to at least 200 amino acids are also preferred. Fragments also include truncated peptides that have x amino acids deleted from the N-terminus and/or C-terminus. In such truncations, x may be 1 or more (ie. 1, 2, 3, 4, 5, 6, 7, 8, 9, 10, 15, 20, 30, 40, 50, 60, 70, 80, 90, 100 or more), but preferably less than 150.

As used herein, the term “antibody” refers to a whole immunoglobulin or its fragment, for example, of the Fab type or (Fab′) 2, which retains antigen-binding activity. It may be a naturally occurring antibody or an antibody obtained by immunization (including, for example, a chimeric antibody or a humanized antibody) or a recombinant antibody constructed using genetic engineering techniques.

In the present description, an antibody or its fragment means an antibody that specifically binds to alpha V integrin or its fragment and does not possess cross-specificity to other proteins (peptides, their fragments) of urine, i.e. is an antibody that has (significantly) greater affinity for integrin alpha V or its fragment than for other proteins (peptides, their fragments) of urine.

In the diagnostic kit of the present invention, antibodies that can be obtained from commercial suppliers can be used, or these antibodies can be obtained by the above or other means.

Without further clarification, it is assumed that on the basis of the above description, an ordinary specialist qualified in this field is able to apply the present invention in its entirety. The following options are illustrative only. They in no way limit the disclosure of the present invention. All cited publications are included as references.

The possibility of an objective manifestation of the technical result when using the invention is confirmed by reliable data given in the examples containing experimental information obtained in the process of conducting research according to the methods adopted in this field. The invention is illustrated by figures.

In medical practice, an analysis based on the identification of tissue-specific markers of the disease is commonly used. This is determined by the fact that only such marker can indicate the development disease in a given organ/tissue. Consequently, “biological specificity” relates to an ability to bind results of the analysis to a specific organ and a specific disease. Only a “biologically specific” analysis makes it possible to identify the disease under investigation in a specific organ.

But the problem of biological specificity is particularly arising during an analysis of blood. This is due to the fact that the circulatory system is common to all internal organs. Accordingly, in a blood test a measured marker must be specific for the disease (so that it is this disease that can be identified) and specific for the organ (so that the organ affected by this disease can be identified).

More than 95% of human prostatic cancers are adenocarcinomas that arise from the epithelial cells that line glands and ducts of the prostate. Consequently, most research on PC has examined changes occurring in the prostatic epithelial cells as they progress from normal to malignant carcinoma cells. However, a growing body of evidence suggests that as a carcinoma develops, changes also occur in the stromal compartment associated with the tumor, and those changes in the tumor microenvironment contribute to tumor progression. Much of the tumor stroma consists of cancer-associated fibroblasts (CAFs) that express a set of proteins distinct from normal fibroblasts and prostatic epithelial cells (Webber J. P., et al., Prostate stromal cell proteomics analysis discriminates normal from tumour reactive stromal phenotypes (Oncotarget, Vol. 7, No. 15 Feb. 25, 2016)). Phenotypically, CAFs closely resemble myofibroblasts exhibiting increased levels of specific marker expression and growth factors when compared to normal prostate fibroblasts. It can be summarized that fibroblasts a) are normal components of numerous tissues, b) during oncogenic tissue degeneration, neighboring fibroblasts demonstrate the properties of myofibroblasts (Kalluri, R., The biology and function of fibroblasts in cancer. Nature Reviews Cancer, volume 16, pages 582-598 (2016)). This statement was the basis of serendipitous hypothesis.

The original working hypothesis was that the tumor markers should not originate explicitly from tumor cells but can also belong to associated myofibroblasts. Oncogenic specificity of the myofibroblast markers lies in the heart of the present invention.

Based on this hypothesis several markers were selected, characterized by the greatest changes. Following the preliminary analysis of their behavior in clinical specimens, the protein ITGaV was selected.

It should be noted that ITGaV, as a marker of the fibroblast/CAF transformation, if detected in blood would lack organ specificity, and thus information about localization of the tumor would be missing. However, in the claimed method, it is urine, not blood, that is analyzed, and it provides a necessary organ specificity.

To sum it up, in the present invention, it was unexpectedly discovered that the quantitative content (level) of integrin alpha V in whole urine correlates with PC, which allows it to be used as a biomarker for the diagnosis of PC, and also made it possible to develop a new method for the diagnosis of PC on the basis of this new biomarker. Quantitative determination of integrin alpha V in whole urine allows non-invasive diagnosis of the disease, and a simple analysis technique allows the proposed method to be used in a standard clinical laboratory of hospitals or medical institutions.

There are well established rules for preparing an urine sample for conventional tests. According to recommendations posted on MedlinePlus, an online curated information service produced by the United States National Library of Medicine (https://medlineplus.gov/encv/article/007487.htm):
before collection, subject should not eat food that can affect the color of urine (beets, carrots, bright berries);
the sample collection container must be sterile, free from traces of cleaning and disinfecting agents. Nowadays, disposable containers are mainly used for these purposes;
urine samples collected for general analysis should be stored for no more than 1.5-2 hours, and always in a cool place, at a temperature of 5-18° C.;
to prevent bacteria from external genital organs from getting into the sample, one need to release a small amount of urine into the toilet, and then, without stopping urination, substitute the container and collect 100-150 ml.

Based on the anatomy of the male urogenital system, prior knowledge and medical practice, the first portion of urine is contaminated with prostate fluids and has to be discarded and not processed for conventional analyses. But urine samples collected by the traditional methods are barely informative and could not provide an adequate data about a presence of prostate cancer in a human subject.

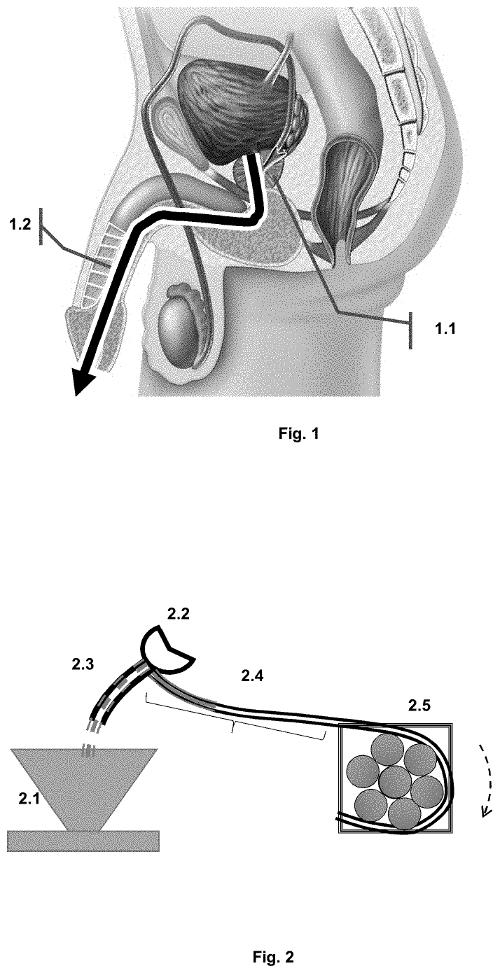

According to the present invention, however, the first portion of urine samples is critical for providing the most informative biomaterial for the method. This is because the first portion of urine contains a washout of secretions of the prostate gland of a subject to the urethral canal (FIG. 1). With a long pause between urination (for example, the first urine after sleep), after a direct prostate massage, after a sexual excitement lasting for more than 5-7 minutes, as well as after ejaculation, the content of the urethral canal is highly enriched with a prostate fluid. Direct evidence of this is the presence of a significant number of spermatozoa in the first portion of urine obtained in the above-described conditions, which are absent in the rest of the urine. Thus, given the anatomical and physiological characteristics of the male body (FIG. 1), it can be argued that the first portion of urine, obtained under well-defined and easily observed conditions, carries data about the state of the prostate gland. It was determined for the claimed method that “the first portion of urine” is up to 30 ml of the first urine during urination. To sum it up, under the specific conditions defined in the claimed method, the marker ITGaV possesses the necessary specificity, i.e. analysis results are related to a) cancer, b) prostate gland. In the preferred realization of the invention, a first morning urine after sleep, called “morning urine”, or “the first portion of urine”, preferentially the first portion of morning urine is used.

### First Portion Significance

The following examples are given to illustrate the method according to the present invention and should not be construed as in any way limiting the scope of the invention.

Following out hypothesis, both full portion of an urine sample and a middle portion of an urine sample in the absence of the separated first portion are not useful. Therefore, it is crucial for the invention implementation to obtain the first portion of a urine sample, separated from the rest of the sample. To study changes in the composition of urine during urination, the authors developed the device shown in FIG. 2.

The contact urinal receiver of the device shown in FIG. 2 is made of a soft polymeric material that does not cause discomfort during a contact with the head of the penis. Urine entering the urinal receiver and moving freely through the main channel is collected in the uroflowmeter, which records the dynamics of fluid accumulation. A flexible polymer tube made of a biologically inert material is attached to a side channel. The speed of a peristaltic pump that regulates operation of the device is chosen so that in the process of urination a convenient for further applications segment of the tube is filled. The resulting sample represents a tube filled with portions of urine distributed along the tube. The entire sample can be frozen and stored at −20° C. To take a sample of the required portion (for example, the first portion), one can measure the corresponding distance along the length of tube and cut a fragment containing a sufficient amount of the sample.

In the method according the invention the most informative data about integrin alpha V levels can be obtained from urine samples gathered either in the morning after sleep (preferentially first morning urine), or after a long (not less than 4 hours) abstention from urination, or 5-10 minutes after sexual stimulation for at least 4-5 minutes, or after a prostate massage. However, the most practical way (and psychologically the most acceptable by subjects) is a collection of the first morning urine, therefore, the data presented in this application were obtained this way.

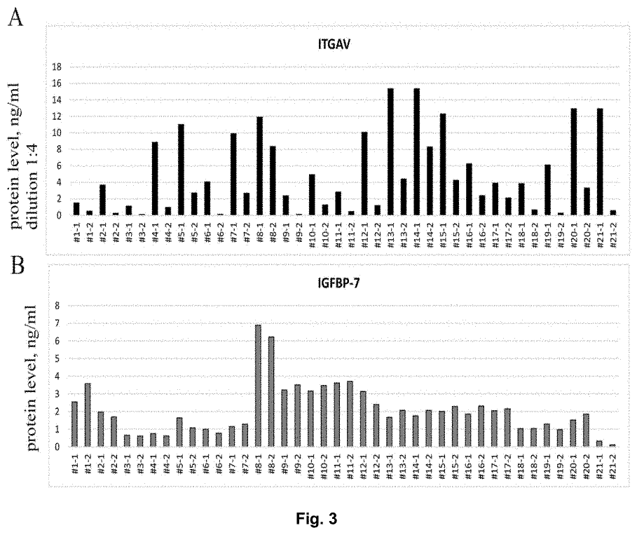

FIG. 3 presents the results of a pilot series of experiments, which compared the level of ITGaV in the blinded samples of first portion of urine with its level in the rest of the urine. Comparison of ITGAV (A) and IGFBP7 (B) concentrations between the first (#n-1) and (#n-2) portions of urine from healthy individuals. The difference in values between the first and second portion of urine for ITGAV is statistically significant (p<0.05) while there is no difference for IGFBP7 protein level (p>0.05) in both portions. The result clearly indicates an increased concentration of ITGAV in the first portion of urine as compared to the second portion of urine. The observation that the level of ITGAV is lower in the second portion of urine presumes false PC prediction if the second portion instead of the first portion of urine is donated.

### Method Description

The invention also covers all kinds of kits and methods for diagnosing prostate cancer. Such kits may include antibodies against integrin alpha V or its fragments, which can be detected both as the individual subunit and/or as a part of heterodimers, such as heterodimeric complexes with beta-1, beta-3, beta-5, beta-6, and/or beta 8. According to the invention any antibody or its fragment, that specifically bind to alpha V integrin and are not cross-specific to other urine peptides can be used. Such antibodies may include, but are not limited to, any antibody/antibody fragments against integrin alpha V known in the prior art (for example, Polyclonal Antibody to Integrin Alpha V (ITGaV) http://www.elabscience.com/Manual/Antibody/EN/ENT2365.pdf, http://cloud-clone.com/products/PAB282Hu01.html, http://www.genetex.com/Integrin-alpha-5-antibody-C-term-GTX81964.html, Monoclonal Antibody to Integrin Alpha V (ITGaV) http://www.abcam.com/integrin-alpha-5-antibody-epr7854-ab150361.html, http://www.sigmaaldrich.com/catalog/product/mm/mabt207, http://cloud-clone.com/products/MAB282Hu22.html, http://www.antibodies-online.com/abstract/Integrin+alpha+V+(ITGAV)+Antibody/) and new, previously unknown antibodies/antibody fragments, including antibodies (or antibody fragments), modified to increase their affinity by known methods, which can be obtained using known methods, such as an immunization method (including, for example, chimeric antibodies or humanized antibodies) or a method for producing recombinant antibodies constructed with using genetic engineering techniques.

The diagnostic kit includes at least one antibody/antibody fragment, specifically binding to integrin alpha V (for example, either immobilized on a substrate, or in a lyophilized form, or as an aqueous solution) and one or more reagents necessary for diagnostic analysis. If an anti-integrin alpha V antibody is labeled with an enzyme, the kit may include substrates and cofactors required for the enzyme activity detection (for example, a precursor substrate that provides a detectable chromophore or fluorophore). In addition, other additives may be included in the kit, such as stabilizers, buffers (for example, blocking buffer) and others. In certain realizations an anti-integrin alpha V antibody included in the diagnostic kit is immobilized on a solid surface or a granular material (for example, a glass slide or plastic plate or a diagnostic strip, etc.). The relative amounts of different reagents can vary widely to ensure concentrations of reagents in the solution that can optimize sensitivity of the assay. In a specific realization an antibody and one or more reagents may be provided (individually or jointly) in the form of dry powders, usually lyophilized, including excipients, which will produce a reagent solution having a suitable concentration if dissolved.

In some realizations, a set for the diagnosis of PC may be a test system of arbitrary design, for example, diagnostic strips, an array, such as a protein microarray (https://en.wikipedia.org/wiki/Protein_microarray), a chip, etc., or a set of reagents for use in manual or automated analysis, or with a partially automatic/fully automatic/robotic analyzer. The reagent kit may also include an IVD device, such as a plate, with immobilized antibodies, which allows for conducting a solid phase ELISA or any other assay with a sensitivity of at least 0.2 ng/ml.

In some realizations of the invention other known from the prior art clinical, laboratory and/or instrumental methods of prostate gland examination may additionally be applied.

In some realizations of the invention, the integrin alpha V biomarker can be used in the diagnosis of PC in combination with another biomarkers that together might improve the quality or accuracy of the diagnosis. These biomarkers include, but are not limited to prostate-specific antigen, PCA3, TMPRSS2:ERG, PSCA (prostate stem cell antigen), AMCAR, and/or others.

### Implementation of the Invention

Example 1. Diagnosis of PC can be carried out by any method of analysis that is able to determine the level of integrin alpha V in the patient's urine. In one particular case, the diagnosis was carried out as follows:
a. about 30 ml of the first portion of morning urine was taken from a subject;
b. the urine sample was clarified by centrifugation for 15 minutes at a speed of 2,000 rpm (filtration through membranes with a pore size of 0.22-0.45 nm or any other available method can be used to separate the soluble fraction of urine proteins from the insoluble sediment), and the sediment was discarded;
c. the level of integrin alpha V was determined in this particular case by ELISA using commercially available kit “SEB282Hu 96 Tests. Enzyme-linked Immunosorbent Assay Kit for Integrin Alpha V (ITGaV)” from Cloud-Clone Corp. (http://cloud-clone.com/products/SEB282Hu.html), by strictly following the instructions (http://cloud-clone.com/manual/ELISA-Kit-for-Integrin-Alpha-V-(ITGaV)-SEB282Hu.pdf).

Briefly:
a. 25 μl. of urine diluted to 100 μl was introduced into a well of a 96-well plate with immobilized antibodies;
b. incubated for 1.5 hours at 37° C.;
c. the liquid was removed and 100 μl of the detection reagent A were added;
d. incubated for 1.5 hours at 37° C.;
e. the liquid was removed and the well was washed 3 times with the washing solution. After each wash, the liquid was carefully removed by shaking out on a filter paper;
f. 100 μl of the detection reagent B were added;
g. the plate was incubated for 0.5 hours at 37° C.;
h. the liquid was removed, and the well was washed 5 times with the washing solution. After each wash, the liquid was carefully removed by shaking out on a filter paper;
i. 90 μl of TMB substrate were added;
j. incubated for 20 min at 37° C.;
k. 50 μl of the stop reagent were added;
l. the absorbance was measured immediately photometrically at 450 nm. To determine the level of ITGaV in each experimental urine sample according to the manufacturer's instructions, a calibration curve obtained from a separate measurement of various dilutions of a standard provided in a kit was used.

At the same time, an increase in the accuracy of analysis and reproducibility of results can be obtained in two ways:
a. due to control of the process of collecting urine: one needs exactly 25-30 ml of the first portion of morning urine without prostate massage;
b. collecting 25-30 ml of the first portion of urine after a prostate massage.
Urine can be stored at +4° C. for a day and for 2-3 weeks at −18° C.
In some cases, it is also advisable to repeat the analysis.

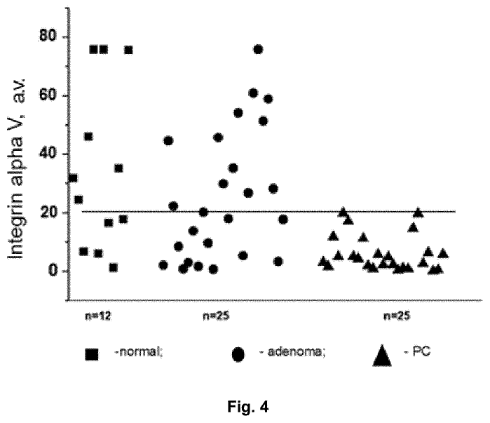

FIG. 4 shows the experimental results of measuring the level of integrin alpha V in the first portion of morning urine of three groups of Caucasians with age over 50 years: healthy people, patients with benign prostatic hyperplasia and patients with prostate cancer. The urine samples were obtained by the above method. A.v.—arbitrary value, the absolute values for the levels of integrin alpha V have not been determined, but only compared between groups.

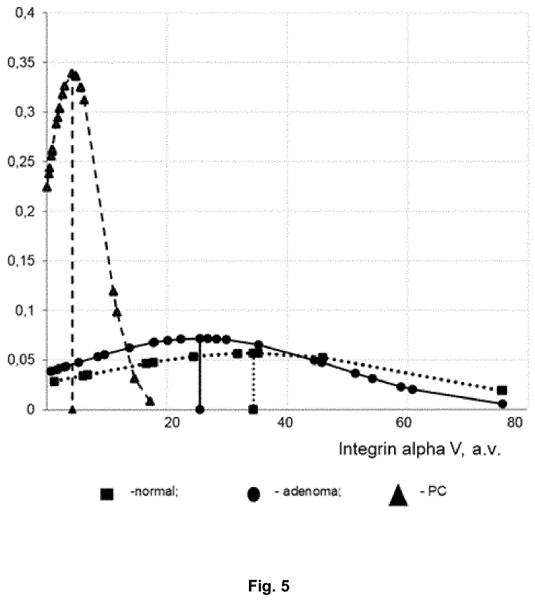

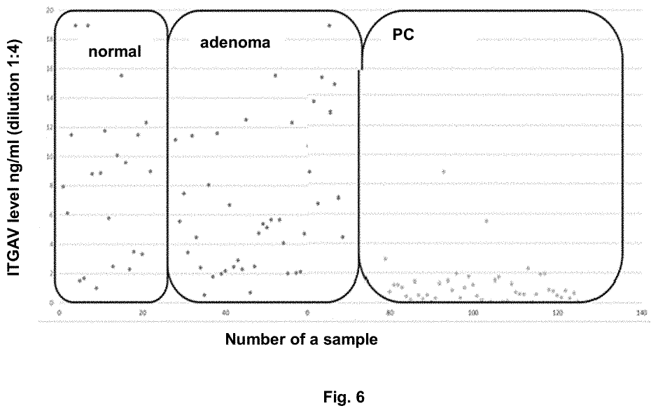

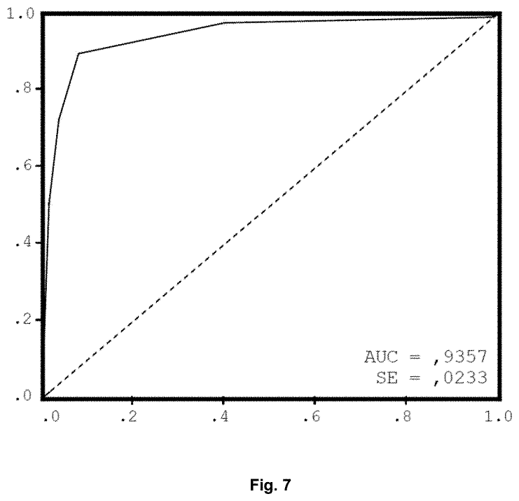

The graph of FIG. 5 shows the curves of the distribution of values for the three data sets and the mathematical expectations of the corresponding measurements (vertical lines). As follows from the graph, the mathematical expectations of the biomarker level in healthy people and patients with benign hyperplasia differ significantly from the mathematical expectation of the biomarker level in patients with prostate cancer). Mathematical processing was carried out under the assumption of a Gaussian distribution of the original data.

As follows from the obtained data the reference value of integrin alpha V concentration in the urine may be established: above this value the patient may be healthy or have prostatic hyperplasia (prostatitis/adenoma); the level of integrin alpha V below this value indicates high probability of PC and should lead to an additional examination of a subject, for example, by means of biopsy and histological analysis.

Example 2. To obtain statistically reliable results, 111 men over 50 years old were examined. After obtaining an informed consent from subjects, the first portion of morning urine samples and necessary information were collected by qualified medical personnel and provided for analysis in an encrypted form (the “Identifier” column in the Table 1). After that, most of the subjects underwent diagnosis by biopsy (urine samples were obtained before taking the biopsy).

Further, the following data is shown in the Table 1. Raw experimental data (dilution 1:4).

TABLE 1
Raw experimental data (dilution 1:4)

| | | | | PSA | PSA | Diagnosis | | | | | ITGVA |
|---|---|---|---|---|---|---|---|---|---|---|---|
| | | Data of | | level | analysis | | | prostate cancer | Diagnostic | | dil 1:4 |
| Number | Identifier | analysis | Age | ng/ml | data | Normal | BPH | Glisson score | data | Treated | ng/ml |
| 1 | U-7 | 14.07.2016 | 80 | 56.6 | 12.07.2016 | | | T3bN0M0G8 | 23.11.2012 | [symbol] | 4.287556 |
| 2 | U-4 | 14.07.2016 | 68 | 11 | 12.07.2016 | | | cT2cN0m0G6(3 + 3) | 11.07.2016 | [symbol] | 2.925622 |
| 3 | U-1 | 14.07.2016 | 65 | 8.6 | 12.07.2016 | | | cT2aN0M0 | 11.07.2016 | [symbol] | 0.750336 |
| 4 | U-5 | 14.07.2016 | 66 | 15.6 | 12.07.2016 | | | cT2bN0M0 | 11.07.2016 | [symbol] | 1.220712 |
| 5 | U-2 | 14.07.2016 | 66 | 1.49 | 13.07.2016 | | | cT2cN0M0 | 2014r | [symbol] | 0.4017 |
| 6 | U-6 | 14.07.2016 | 65 | 9.47 | 14.07.2016 | | | T2N0M0 | 13.07.2016 | | 4.933964 |
| 7 | U-8 | 25.07.2016 | 76 | 23 | 15.07.2016 | | | cT3bN0M0 | 15.07.2016 | | 1.221289 |
| 8 | U-3 | 14.07.2016 | 75 | 1.47 | 15.05.2016 | | x | | 14.07.2016 | | 0.5044 |
| 9 | U-9 | 15.07.2016 | 58 | 1.1 | 16.07.2016 | x | | | 14.07.2016 | | 7.932052 |
| 10 | U-10 | 16.07.2016 | 58 | 1.1 | 17.07.2016 | x | | | 14.07.2016 | | 6.102131 |
| 11 | U-11 | 18.07.2016 | 65 | 20 | 17.05.2016 | | | (3 + 4); cT2aN0M0 | 15.07.2016 | [symbol] | 1.050467 |
| 12 | U-12 | 19.07.2016 | 63 | 0.9 | 19.07.2016 | | | [symbol] | 18.07.2016 | [symbol] | 2.774741 |
| 13 | U18 | 19.07.2016 | 53 | 2.24 | 19.07.2016 | | x | | 18.07.2016 | | 11.13399 |
| 14 | U-14 | 19.07.2016 | 64 | 20.65 | 18.07.2016 | | | (3 + 4); T3bN0M0 | 18.07.2016 | [symbol] | 0.449944 |
| 15 | U-19 | 19.07.2016 | 65 | 5.43 | 18.07.2016 | | | (4 + 3); T2aN0M0 | 18.07.2016 | [symbol] | 0.220954 |
| 16 | U-20 | 21.07.2016 | 58 | 10.89 | 19.07.2016 | | | (3 + 3)T2cN0M0 | 19.07.2016 | [symbol] | 1.407548 |
| 17 | U-15 | 26.07.2016 | 51 | 1.04 | 26.07.2016 | x | | | 25.07.2016 | | 11.47849 |
| 18 | U-16 | 26.07.2016 | 66 | 1.19 | 26.07.2016 | x | | | 25.07.2016 | | 18.93675 |
| 19 | U-21 | 28.07.2016 | 54 | 0.94 | 27.07.2016 | | x | | 26.07.2016 | | 5.559008 |
| 20 | U-22 | 28.07.2016 | 63 | 1.57 | 27.07.2016 | | x | | 26.07.2016 | | 7.459038 |
| 21 | U-24 | 28.07.2016 | 58 | 2.93 | 28.01.2016 | | x | | 26.07.2016 | | 2.110146 |
| 22 | U-25 | 28.07.2016 | 53 | 9 | 16.04.2016 | | | (3 + 3) T2N0M0 | 26.07.2016 | [symbol] | 0.519852 |
| 23 | U-23 | 29.07.2016 | 66 | 48.4 | 16.06.2016 | | | (4 + 3) T2cN0M0 | 28.07.2016 | [symbol] | 0.232748 |
| 24 | U-17 | 02.08.2016 | 56 | 2.33 | 01.08.2016 | x | | | 01.08.2016 | | 1.520042 |
| 25 | U-26 | 02.08.2016 | 79 | 3.2 | 21.07.2016 | | x | | 01.08.2016 | | 0.191947 |
| 26 | U-29 | 02.08.2016 | 68 | 4.09 | 28.07.2016 | | x | | 01.08.2016 | | 0.732251 |
| 27 | U-30 | 02.08.2016 | 68 | 3.29 | 06.05.2016 | | x | | 01.08.2016 | | 3.427777 |
| 28 | U-27 | 02.08.2016 | 70 | 0.89 | 13.06.2016 | | | (3 + 3) T2bN0M0 | 25.03.2015 | [symbol] | 1.198927 |
| 29 | U-28 | 03.08.2016 | 69 | 28.6 | 28.07.2016 | | | (3 + 4) cT2cN0M0 | 02.08.2016 | [symbol] | 0.529526 |
| 30 | U-13 | 05.08.2016 | 56 | 1.6 | 12.07.2016 | x | | | 04.08.2016 | | 1.678438 |
| 31 | U-31 | 23.08.2016 | 62 | 9.3 | 12.07.2016 | | | (4 + 4)cT3bN0M0 | 22.08.2016 | [symbol] | |
| 32 | U-32 | 24.08.2016 | 7 | 6.4 | 19.08.2016 | | | (4 + 5)cT3bN1M1b | [symbol] | [symbol] | |
| 33 | U-33 | 24.08.2016 | 65 | 24 | 08.07.2016 | | | (3 + 4)cT2cN0M0 | 23.08.2016 | [symbol] | 5.024719 |
| 34 | U-34 | 29.08.2016 | 61 | 3.12 | 26.08.2016 | x | | | 22.08.2016 | | 18.93675 |
| 35 | U-35 | 30.08.2016 | 65 | 6.18 | 22.07.2016 | x | | | c 2005 | | 4.12272 |
| 36 | U-43 +/ [symbol] | 31.08.2016 | 85 | 8.2 | 08.08.2016 | | x | | 26.07.2016 | | 11.41518 |
| 37 | U-44 +/ [symbol] | 31.08.2016 | 59 | 0.92 | 26.08.2016 | x | | | 26.08.2016 | | 8.778977 |

[symbol]

indicates data missing or illegible when filed

To “clean up” the data, we excluded from further analysis those patients who had previously undergone any anti-cancer treatment procedures. After “cleaning” the data from the information on “treated” patients, all other data were summarized in a single diagram presented in FIG. 6, which presents the results of measuring the ITGaV level in the first portion of morning urine from patients who did not undergo anti-cancer therapy or surgery.

The chart demonstrates significant difference between the level of ITGaV in the urine of healthy patients/patients with BPH and from the patients diagnosed with PC.

### Statistical Characteristics of the Diagnostic Test

The most important characteristics of the diagnostic method include:
i. Sensitivity (Se),
ii. Specificity (Sp).

Sensitivity (Se)—defined as the proportion of correct results about the presence of the disease among all patients who have a reliable diagnosis. Sensitivity shows the proportion of patients with the disease that will receive the correct result (diagnosis) based on the claimed method. To determine sensitivity, results of the developed method were compared with the results of another method adopted as the “gold standard”, biopsy data (histology) or other data. The data of the gold standard or biopsy was a criterion for determining the actual presence or absence of the disease.

Se was determined by the formula:

$$S e = \frac{T P}{{D}^{-}} \times 100 \%$$

i. where
ii. D⁻—total number of patients (confirmed by biopsy),
iii. TP—the number of correct results for PC diagnosis (from D⁻).

In the claimed method based on data from Example 2:

Se=43/47×100%=91.5% 1.

Specificity of the method (Sp) characterizes the ability of the diagnostic method not to give false-positive results. Sp is defined as the proportion of negative results among healthy individuals in the group studied and is determined by the formula:

$$S p = \frac{T N}{D} \times 100 \%$$

i. where
ii. D—total number of healthy persons,
iii. TN—the number of correct diagnostic results of the absence of PC.

In the claimed method based on data from Example 2:

$$Sp = \frac{64}{70} \times 100 \% = 91 , 4 \%$$

The big set of the main statistical indicators of the proposed invention were obtained using the freely distributed Diagnostic Utility Statistics software by Marley W.

Watkins, created to calculate the effectiveness of diagnostic solutions:

| | |
|---|---|
| Sensitivity (true positive rate) | 0.9149 |
| Specificity (true negative rate) | 0.9143 |
| False Positive Rate | 0.0857 |
| False Negative Rate | 0.0851 |
| Positive Predictive Power | 0.8776 |
| Proportion Misleading Positives | 0.1224 |
| Incremental Validity of Positive Test Diagnoses. | +0.4758 |
| Quality of the Positive Predictive Power | +0.7953 |
| Negative Predictive Power | 0.9412 |
| Proportion Misleading Negatives | 0.0588 |
| Incremental Validity of Negative Test Diagnoses. | +0.3429 |
| Quality of the Negative Predictive Power | +0.8536 |
| Odds Ratio | +114.67 |
| Likelihood Ratio(+) | +10.674 |
| Likelihood Ratio(−) | +0.0931 |
| Diagnostic Odds Ratio | +114.67 |
| Prevalence (base rate) | 0.4017 |
| Error rate | 0.0855 |
| Observed Agreement (hit rate/accuracy) | 0.9145 |
| Chance Agreement | 0.5160 |
| Kappa | +0.8234 |
| Standard Error of Kappa | .0924 |
| Z | +8.9122 |
| P | .0000 |
| G | +0.8291 |
| Phi coefficient | +0.8239 |
| Absolute Risk Reduction (ARR) | 0.8187 |
| Youden's J Index | 0.8187 |
| Number Needed to Treat | 1 |
| Conditional Probabilities | |
| Disorder Present Given Positive Test (PPP) | 0.8776 |
| Disorder Present Given Negative Test | .0588 |
| Disorder Absent Given Positive Test | 0.1224 |
| Disorder Absent Given Negative Test (NPP) | 0.9412 |
| Positive Test Given Disorder Present (Sen) | 0.9149 |
| Positive Test Given Disorder Absent (FPR) | 0.0857 |
| Negative Test Given Disorder Present (FNR) | 0.0851 |
| Negative Test Given Disorder Absent (Spc) | 0.9143 |

The presented data indicate that integrin alpha V can be used as a new biomarker for the diagnosis of prostate cancer, and its determination in the urine can be used for non-invasive diagnosis of prostate cancer, as well as for developing a simple, sensitive, reliable method suitable for clinical laboratory for diagnosis of prostate cancer. The proposed diagnostic method can be used for population surveys, monitoring of risk groups, monitoring of relapses, monitoring the progress of the disease, monitoring of therapy.

Although the invention has been described with a reference to the disclosed embodiments, it should be obvious to those skilled in the art that the specific experiments described in detail are given only for the purpose of illustrating the present invention and should not be construed as limiting the scope of the invention in any way. It should be clear that it is possible to implement various modifications without departing from the gist of the present invention.

1. A noninvasive method for distinguishing prostate cancer from non-cancer in a human subject, comprising:
obtaining a sample of a first portion of a morning urine from the subject;
determining a level of integrin alpha V or its fragments in said sample;
comparing said level with a reference value, obtained from urine samples collected from non-cancer subjects and processed in essentially the same way as the subject's sample;
predicting a presence of prostate cancer in said subject when said level is lower than said reference value;
predicting an absence of prostate cancer in said subject when said level is higher than said reference value.

2. The noninvasive method according to claim 1, wherein the level of integrin alpha V or its fragments was determined in the first portion of the morning urine from the subject without additional processing of said first portion.

3. The noninvasive method according to claim 1, wherein the level of integrin alpha V or its fragments is determined by an enzyme-linked immunosorbent assay.

4. The noninvasive method according to claim 3, wherein the first portion comprises between 25-30 ml of the first morning urine.

5. The noninvasive method according to claim 4, wherein the human subject is a subject who was never treated for prostate cancer.

6. The noninvasive method according to claim 5, wherein the human subject is a Caucasian and the reference value is 8 ng/ml.

7. The noninvasive method according to claim 5, wherein sensitivity and specificity for distinguishing prostate cancer from non-cancer are both greater than 90%.

8. The noninvasive method according to claim 1, wherein the level of integrin alpha V is determined as a level of an individual subunit or as a level of one of the integrin heterodimers selected from the following group: αvβ1, αvβ3, αvβ5, αvβ6, αvβ8.

9. A method for treating prostate cancer in a human subject, comprising:
obtaining a sample of a first portion of a morning urine from the subject;
determining a level of integrin alpha V or its fragments in said sample;
comparing said level with a reference value, obtained from urine samples collected from non-cancer subjects and processed in essentially the same way as the subject's sample;
when said level is less than said reference value, referring said subject to one or more additional methods for prostate cancer verification or classification;
treating the subject by one or more available methods based on the verification or classification outcome.

10. The method for treating prostate cancer according to claim 9, wherein determining the level of integrin alpha V or its fragments in the sample occurs without lysis or fractionation of extracellular vesicles from said sample.

11. The method for treating prostate cancer according to claim 10, wherein the level of integrin alpha V or its fragments is determined by an enzyme-linked immunosorbent assay.

12. The method for treating prostate cancer according to claim 11, wherein the first portion comprises between 25-30 ml of the first morning urine.

13. The method for treating prostate cancer according to claim 12, wherein the human subject is a subject who was never treated for prostate cancer.

14. The method for treating prostate cancer according to claim 13, wherein the human subject is a Caucasian and the reference value is 8 ng/ml.


SEQUENCE LISTING (1 sequence)

SEQ ID NO: 1 - protein, 1018 aa, Homo sapiens
FNLDVDSPAEYSGPEGSYFGFAVDFFVPSASSRMFLLVGAPKANTTQPGIVEGGQVLKCDWSSTRRCQPIEFDATGNRDYAKDDPLEFKSHQWFGASVRSKQDKILACAPLYHWRTEMKQEREPVGTCFLQDGTKTVEYAPCRSQDIDADGQGFCQGGFSIDFTKADRVLLGGPGSFYWQGQLISDQVAEIVSKYDPNVYSIKYNNQLATRTAQAIFDDSYLGYSVAVGDFNGDGIDDFVSGVPRAARTLGMVYIYDGKNMSSLYNFTGEQMAAYFGFSVAATDINGDDYADVFIGAPLFMDRGSDGKLQEVGQVSVSLQRASGDFQTTKLNGFEVFARFGSAIAPLGDLDQDGFNDIAIAAPYGGEDKKGIVYIFNGRSTGLNAVPSQILEGQWAARSMPPSFGYSMKGATDIDKNGYPDLIVGAFGVDRAILYRARPVITVNAGLEVYPSILNQDNKTCSLPGTALKVSCFNVRFCLKADGKGVLPRKLNFQVELLLDKLKQKGAIRRALFLYSRSPSHSKNMTISRGGLMQCEELIAYLRDESEFRDKLTPITIFMEYRLDYRTAADTTGLQPILNQFTPANISRQAHILLDCGEDNVCKPKLEVSVDSDQKKIYIGDDNPLTLIVKAQNQGEGAYEAELIVSIPLQADFIGVVRNNEALARLSCAFKTENQTRQVVCDLGNPMKAGTQLLAGLRFSVHQQSEMDTSVKFDLQIQSSNLFDKVSPVVSHKVDLAVLAAVEIRGVSSPDHVFLPIPNWEHKENPETEEDVGPVVQHIYELRNNGPSSFSKAMLHLQWPYKYNNNTLLYILHYDIDGPMNCTSDMEINPLRIKISSLQTTEKNDTVAGQGERDHLITKRDLALSEGDIHTLGCGVAQCLKIVCQVGRLDRGKSAILYVKSLLWTETFMNKENQNHSYSLKSSASFNVIEFPYKNLPIEDITNSTLVTTNVTWGIQPAPMPVPVWVIILAVLAGLLLLAVLVFVMYRMGFFKRVRPPQEEQEREQLQPHENGEGNSET